(19) United States
(12) Patent Application Publication
(10) Pub. No.: US 2010/0016465 A1
(43) Pub. Date: Jan. 21, 2010

(54) Composite Material Comprising A Water-Or Acid-Releasing Agent

(76) Inventors: Alexander Van Lelieveld, Frederiksberg (DK); Mette Skovgaard Nielsen, Taastrup (DK); Kristoffer Almdal, Roskilde (DK); Søren Linderoth, Roskilde (DK)

Correspondence Address: HARNESS, DICKEY & PIERCE, P.L.C., P.O. BOX 8910, RESTON, VA, 20195, US

(21) Appl. No.: 12/225,065

(22) PCT Filed: Mar. 13, 2007
(86) PCT No.: PCT/DK2007/000124

§ 371 Date: Mar. 13, 2009

(30) Foreign Application Priority Data
Mar. 13, 2006 (DK) PA 2006 00353
Dec. 22, 2006 (DK) PA 2006 01707

(51) Int. Cl. A61K 6/08

(52) U.S. Cl. 523/116

(57) ABSTRACT

The present invention discloses a composite material comprising one or more fillers and a polymerizable resin base, wherein said one or more fillers comprise at least one filler ingredient, said filler ingredient(s) being present in a metastable first phase and being able to undergo a martensitic transformation to a stable second phase, the volume ratio between said stable second phase and said metastable first phase of said filler ingredient(s) being at least 1.005, and wherein said material further comprises one or more water- or acid- releasing agents; as well as a corresponding method of controlling the volumetric shrinkage of a composite material upon curing.

### FIELD OF THE INVENTION

The present invention relates to composite materials comprising a water- or acid-releasing agent.

### BACKGROUND OF THE INVENTION

The applicant's earlier PCT application No. WO 2005/099652 Al discloses a composite material exhibiting a low or even negligible volumetric shrinkage upon curing, or even a small expansion (e.g. up to 0.5%), in particular composite materials in the form of dental filling materials, as well as a method of controlling volumetric shrinkage of a composite material upon curing. According to WO 2005/099652 A1, a volume stable composite material for dental use can, e.g., be obtained by the use of metastable zirconia particles. Since a volume stable composite can minimize crack formation, such a technology is of great commercial importance. It was suggested that the martensitic transformation of such composite materials could be activated either by physical means or by chemical means (e.g. modification of the surface free energy by contacting the surface of the filler ingredient particles with a chemical, e.g. a constituent of the composite material or an additive such as water).

However, in order to obtain the best possible properties, it is desirable to more accurately control the transformation process, e.g. by means of more refined chemical means.

### BRIEF DESCRIPTION OF THE INVENTION

One aspect of the present invention relates to a composite material comprising one or more fillers and a polymerizable resin base, wherein said one or more fillers comprise at least one filler ingredient, said filler ingredient(s) being present in a metastable first phase and being able to undergo a martensitic transformation to a stable second phase, the volume ratio between said stable second phase and said metastable first phase of said filler ingredient(s) being at least 1.005, and wherein said material further comprises one or more water- and/or acid-releasing agents.

Another aspect of the present invention relates to a composite material comprising one or more fillers and a polymerizable resin base, wherein said one or more fillers comprise at least one filler ingredient, said filler ingredient(s) including metastable zirconia in the tetragonal or cubic crystalline phase, wherein said resin base, upon polymerization and in the absence of any compensating effect from the one or more filler ingredients, causes a volumetric shrinkage (ΔVᵣₑₛᵢₙ) of the composite material of at least 0.50%, and wherein said composite material, upon polymerization of said resin base and upon phase transformation of said filler ingredient(s), exhibits a total volumetric shrinkage (ΔVₜₒₜₐₗ) of at least 0.25%-point less than the uncompensated volumetric shrinkage (ΔVᵣₑₛᵢₙ) caused by the resin base, and wherein said material further comprises one or more water- or acid-releasing agents.

The invention further relates to a method of controlling the volumetric shrinkage of a composite material upon curing, comprising the step of:

(a) providing a composite material comprising one or more fillers and a polymerizable resin base, wherein said one or more fillers comprise at least one filler ingredient, said filler ingredient(s) being present in a metastable first phase and being able to undergo a martensitic transformation to a stable second phase, the volume ratio between said stable second phase and said metastable first phase of said filler ingredient(s) being at least 1.005, and wherein said material further comprises one or more water- or acid-releasing agents;

(b) allowing the resin base to polymerize and cure, and allowing the filler ingredient(s) to undergo a martensitic transformation from said first metastable phase to said second stable phase.

Moreover, the present invention provides the composite materials defined herein for use in medicine, in particular in dentistry.

### DETAILED DESCRIPTION OF THE INVENTION

The present invention, i.a., provides a composite material with improved control of the volumetric shrinkage upon curing of the material.

More particularly, the present invention provides composite material comprising one or more fillers and a polymerizable resin base, wherein said one or more fillers comprise at least one filler ingredient, said filler ingredient(s) being present in a metastable first phase and being able to undergo a martensitic transformation to a stable second phase, the volume ratio between said stable second phase and said metastable first phase of said filler ingredient(s) being at least 1.005, and wherein said material further comprises one or more water- or acid-releasing agents.

A particular feature of the present invention is the presence of one or more water- or acid-releasing agents. The one or more water- or acid-releasing agents represent a well-controlled chemical trigger mechanism with the purpose of contributing to the martensitic transformation of the filler ingredient(s) (see further below).

It is well known that many polymeric resin bases (see also below) exhibit volumetric shrinkage upon curing thereof. Thus, a particular feature of the present invention is the presence of a filler ingredient that will reduce or eliminate the volumetric shrinkage caused by the polymerizable resin base, or even counteract this volumetric shrinkage to such an extent that the composite material exhibits a net volumetric expansion upon curing of the polymeric resin base.

Thus, in a preferred embodiment of the composite material, the resin base, upon polymerization and in the absence of any compensating effect from the one or more filler ingredients, causes a volumetric shrinkage (ΔVᵣₑₛᵢₙ) of the composite material of at least 0.50%, and wherein said composite material, upon polymerization of said resin base and upon phase transformation of said filler ingredient(s), exhibits a total volumetric shrinkage (ΔVtₜₒₜₐₗ) of at least 0.25%-point less than the uncompensated volumetric shrinkage (ΔVᵣₑₛᵢₙ) caused by the resin base. More particularly, the volumetric shrinkage (ΔVᵣₑₛᵢₙ) is at least 1.00%, such as at least 1.50%, and the total volumetric shrinkage (ΔVₜₒₜₐₗ) is at least 0.50%-point less, such as 1.00%-point less than the uncompensated volumetric shrinkage, and wherein said material further comprises one or more water- or acid-releasing agents.

Alternatively, the present invention provides a composite material comprising one or more fillers and a polymerizable resin base, wherein said one or more fillers comprise at least one filler ingredient, said filler ingredient(s) including metastable zirconia in the tetragonal or cubic crystalline phase, wherein said resin base, upon polymerization and in the absence of any compensating effect from the one or more filler ingredients, causes a volumetric shrinkage (ΔVᵣₑₛᵢₙ) of the composite material of at least 0.50%, and wherein said composite material, upon polymerization of said resin base and upon phase transformation of said filler ingredient(s), exhibits a total volumetric shrinkage (ΔVₜₒₜₐₗ) of at least 0.25%-point less than the uncompensated volumetric shrinkage (ΔVᵣₑₛᵢₙ) caused by the resin base, and wherein said material further comprises one or more water- or acid-releasing agents.

The composite material typically comprises 5-95%, or 10-90%, by weight of the one or more fillers (including nanofillers and filler ingredient(s)) and 5-95%, or 10-90%, by weight of the polymerizable resin base, in particular 30-95%, or 30-90%, by weight of the one or more fillers and 5-70%, or 10-70%, by weight of the polymerizable resin base.

The one or more water- or acid-releasing agent typically constitute 0.01-5% by weight, e.g. 0.1-1% by weight, of the composite material.

Preferably, the composite material is substantially solvent free and water free. By the term “substantially solvent free and water free” is meant that the composite material comprises less than 1%, such as less than 0.5% or less than 150 ppm, by weight of solvents and/or water.

Water- or Acid-Releasing Agents (Chemical Triggers)

The one or more water- and/or acid-releasing agents play a role as chemical trigger(s) in the composite materials, i.e. the water-releasing agent will—upon release of water or acid—contribute to or even be solely responsible for the triggering of the martensitic transformation of the filler ingredient(s).

The acids of relevance as chemical triggers are proton-releasing molecules, preferably small molecules like HCl, HF and HBr.

In a particularly interesting embodiment, the chemical triggering is effected by a combination of water and an acid, i.e. the composite material comprises a combination at least one water-releasing agent and at least one acid-releasing agent.

Examples of water- or acid-releasing agents are those which, e.g., under the influence of light or heat, decompose or condense by the simultaneous liberation of water or an acid.

A particular preferred application is a light induced release of water or a strong acid or a combination of both, since the curing of dental composites most commonly is done by light (blue).

Advantageously, the phase transformation takes place along with the curing of the composite material, in particular dental material. It is believed that this can be achieved by a number of water- or acid-releasing agents, e.g. agents releasing water or acid as a result of

A. Aldol/Clalsen Condensations

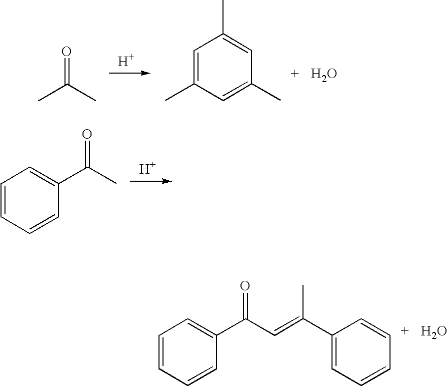

B. Cyclodehydration

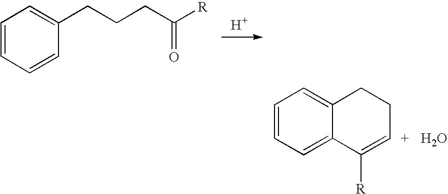

C. Amino-alkylation (the Mannich-Reaction)

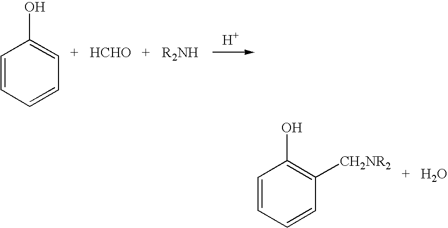

D. Formation of Acetals

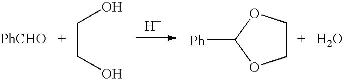

E. Pinacol-Rearrangement

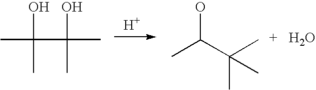

F. Hydroxyalkyl-hydroxy-Elimination

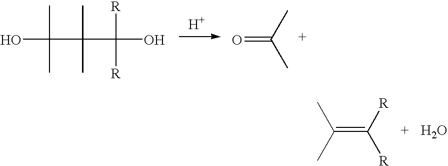

G. Hydrazines in Condensation Reactions

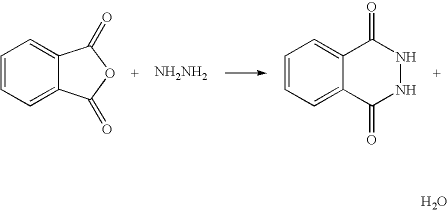

H. Cleavage of Quaternary Ammonium Hydroxides

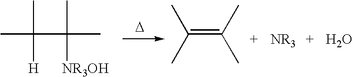

I. Hydro-Peroxides

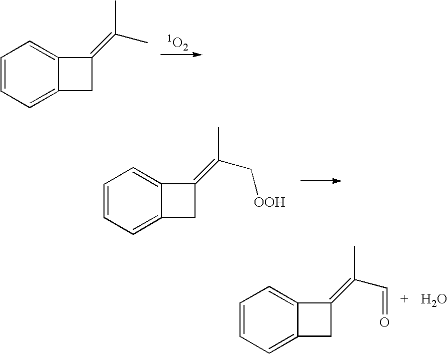

J. Condensation of Nitro-Functionalized Molecules

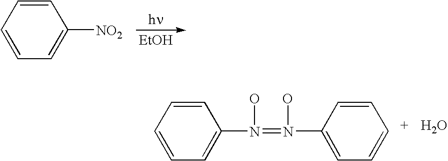

K. A Number of Molecules can Undergo an Elimination to form Water and a Double Bond upon Exposure to Light, some Examples are given below:

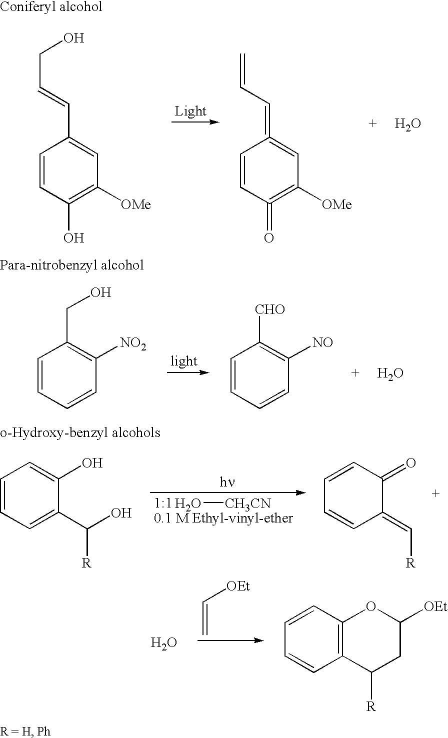

Examples hereof are given, e.g., in J. Am. Chem. Soc. 117 (1995) 5369, J. Org. Chem. 68 (2003) 9643, J. Am. Chem. Soc.123 (2001) 8089, and J. Org. Chem. 66 (2001) 41.

L. Acidity of Photo-Excited Hydroxyarenes

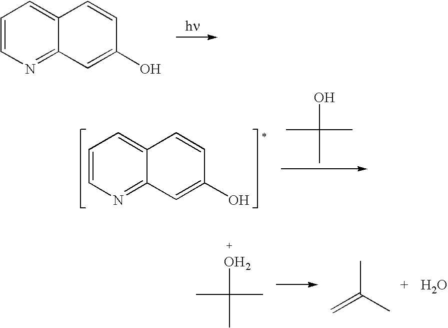

M. Acid-Releasing Photoacids

Ref.: Acc. Chem. Res. 35 (1999) 19 and J. Am. Chem. Soc. 116 (1994) 10593: 5-cyano-1-naphthol; 5,8-dicyano-1-naphthol; 5-, 6-, 7-, and 8-cyano-2-naphthols; 5,8-dicyano-2-naphthol and 5-(methanesulfonyl)-1-naphthol.

Ref.: Luminescence 20 (2005) 358: 7-hydroxy-1-naphthalenesulphonic acid.

Ref.: Tetrahedron Lett. 46 (2005) 5563: Anthracene-9-methanol derived esters.

N. Acid-Forming Agents

Metastable zirconia particles can be phase transformed by HCl soluted in iso-propanol. A hydrohalogen compound could be used to release hydrohalogen upon light radiation. This could be done in a resin base with the metastable zirconia filler particles.

O. Acid-Releasing Esterification

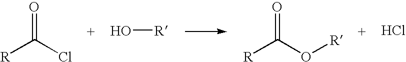

P. Halogen Containing Photo-Acids

In principle it is possible to use halogen-containing radiation-sensitive compounds which form hydrohalogenic acid from any organic halogen compound. The following illustrative halogen-containing organic compounds can be found useful as triggers: carbon tetrabromide, tetra(bromomethyl)-methane: tetrabromoethylene; 1,2,3,4-tetrabromobutane; trichloroethoxyethanol; p-iodophenol; p-bromophenol; p-iodo-biphenyl; 2,6-dibromophenol; 1-bromo-2-naphthol; p-bromoaniline; hexachloro-p-xylene; trichloriacetanilide; p-bromodimethylaniline; tetrachloritetrahydronaphthalene; α,α′-dibromozylene, α,α,α′,α′-tetrabromoxylene; hexabromoethane; 1-chloroanthraquinone; ω,ω,ω-tribromoquinaldine; hexabromocyclohexane; 9-bromofluorene; bis(pentachloro)cyclopentadienyl. It appears probable that halogen radicals are produced from the halogen containing triggers upon adequate radiation. The radicals then react with hydrogen atoms available from a hydrogen donor component to form hydrogen halide which then serves as trigger molecule upon reaction with the zirconia particles.

A currently highly preferred subgroup of the halogen containing compounds are compounds containing a triazine group, in particular triazine compounds comprising one or two trihalomethyl groups represented by the following general formula (I):

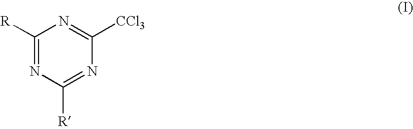

wherein CCl₃ may be replaced by a CF₃ group; R represents the attachment point for an organic moiety; and R′ is selected from the group consisting of a hydrogen atom, a trihalomethyl group (e.g. trichloromethyl group or a trifluoroalkyl group), a substituted C₁₋₆-alkyl group, an unsubstituted C₁₋₆-alkyl group, a substituted aryl group, an unsubstituted aryl group, and a substituted C₂₋₆-alkenyl group.

Preferred examples of the substituent R′ are trichloromethyl and trifluoromethyl groups Examples of the organic moiety R include 4-styrylphenyl and 4-(substituted)-styrylphenyl groups, these molecules typically absorb UV-light.

Illustrative examples of useful mono- or di-trihalomethyl-triazine compounds are those disclosed EP 0 563 925 A1:

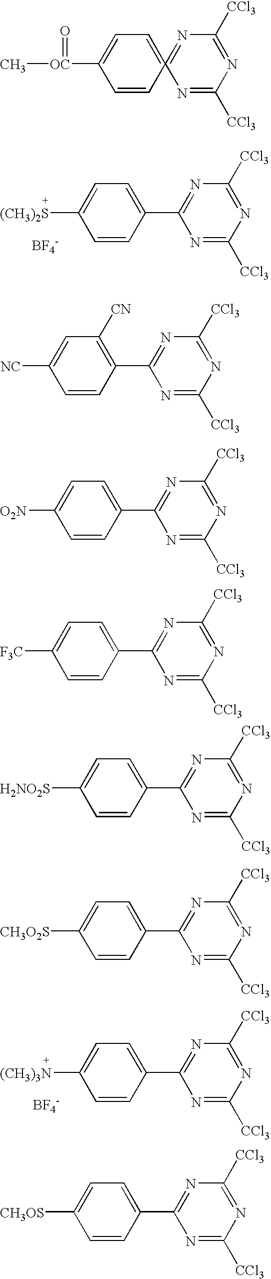

Other useful examples of the organic moiety are styryl and substituted styryl groups, cf. U.S. Pat. No. 3,987,037 and J. Am. Chem. Soc. 121 (1999) 6167, because of the C₂₋₆-alkenyl group these molecules absorb UV-light and sometimes blue light making them more preferable in dental applications;

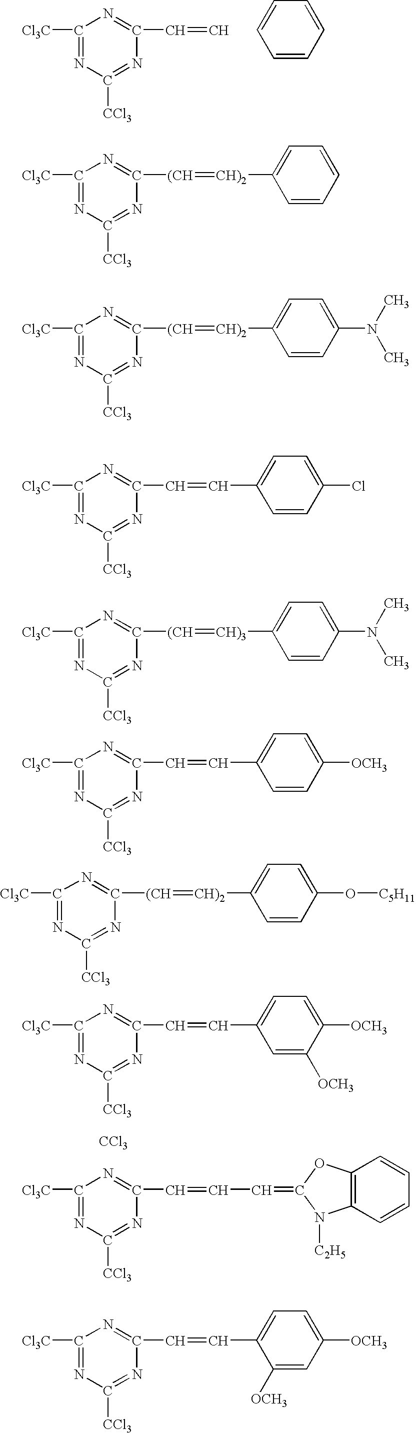

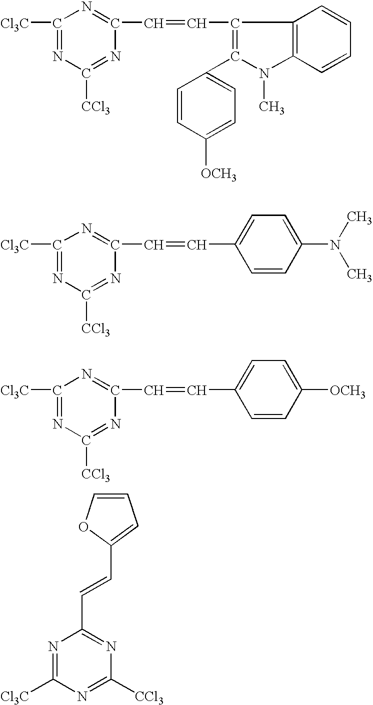

Moreover, other currently preferred examples of organic moieties are residues of polynuclear aromatic compounds such as naphthyl group and residues of heteroaromatic compounds such as thiofuran, J. Am. Chem. Soc. 121 (1999) 6167.

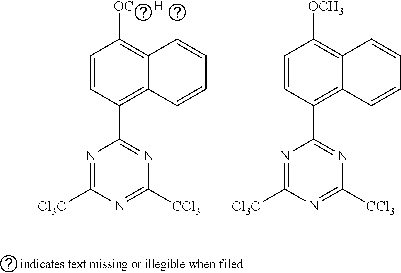

Examples of functional groups required for forming a coupling with the light absorbing moiety S include those listed above in connection with the compounds shown below cf. the compounds disclosed in U.S. Pat. No. 5,262,276. By designing the moiety S the trigger molecules can be “tuned” to the wavelength that is usually used in dental applications (blue light). Furthermore these molecules are reported to bleach upon exposure to light this making them ideal to dental applications were the color of the composite is very important.

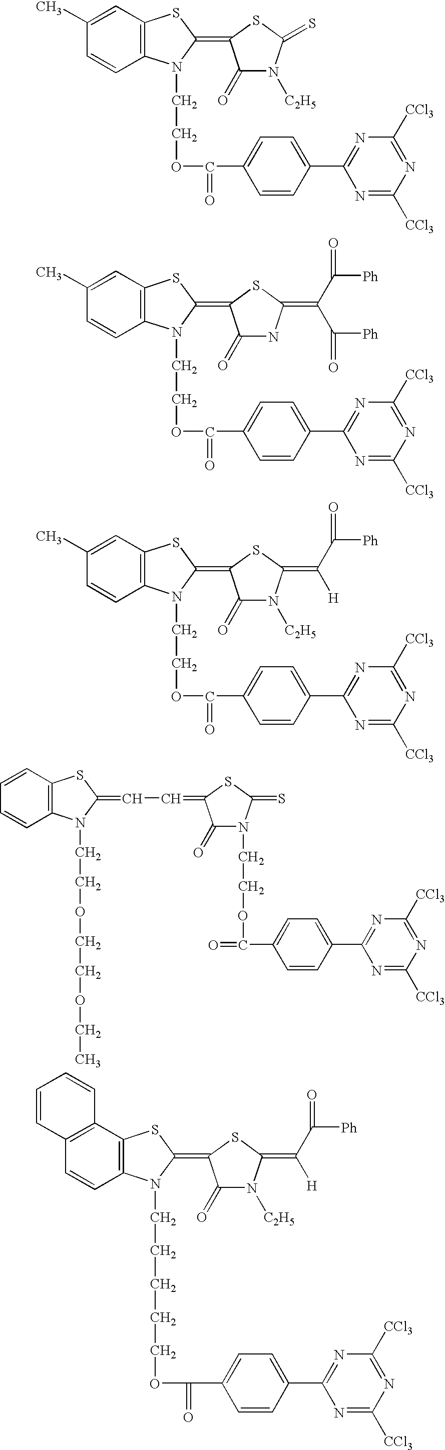

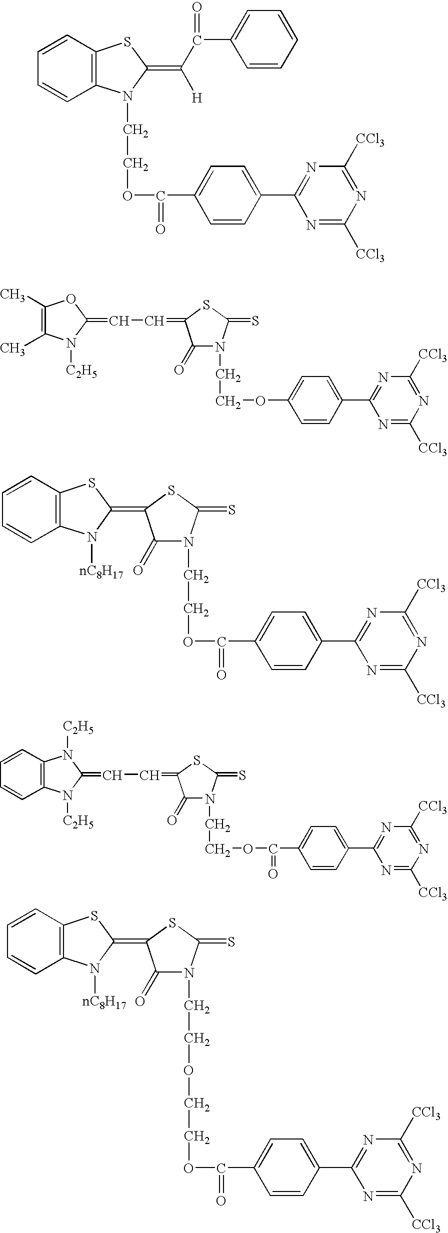

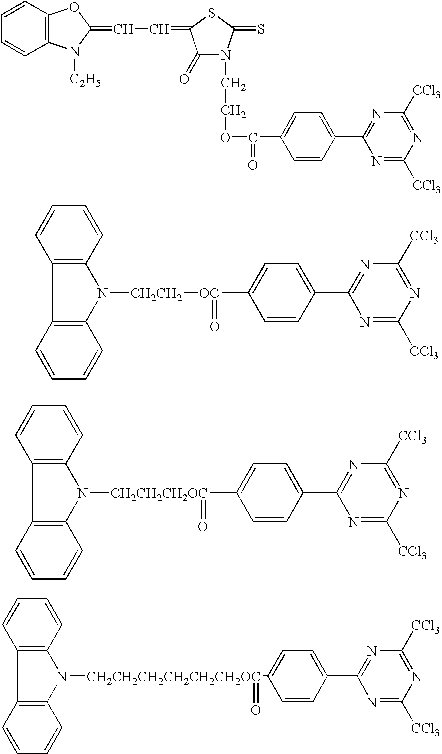

It is envisaged that a further improved application of the above-mentioned triggers would be to chemically anchor the triggers to the surface of the metastable zirconia particles. This would ensure that the triggering molecules were close to the reactive sites of zirconia thereby inducing a fast phase-transformation and reducing the risk of other chemical reactions e.g. with the monomer resin. The chemical anchoring could be done with the use of a silane-, phosphate-, carboxylic acid, hydroxamic acid or a carbamate- group and done with surface treatment of the zirconia particles. E.g.:

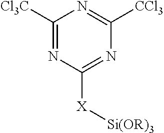

In the formula, X represents an organic moiety as described and illustrated above. In some embodiments, X independently is selected from a substituted C₁₋₆-alkyl group, an unsubstituted C₁₋₆-alkyl group, a substituted aryl group, an unsubstituted aryl group, and a substituted C₂₋₆-alkenyl group, provided that X must have at least one functional group which absorbs light. R in Si(OR)₃ is a substituted or unsubstituted C₁₋₆-alkyl group, typically methyl.

Examples of silane containing novel light-free radical generator compounds which may be used in the present invention are as follows but the present invention is not restricted to these specific examples:

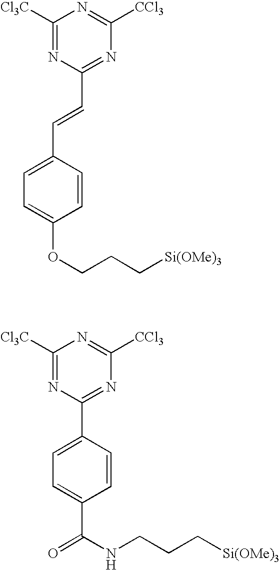

Another way to chemically anchor the triggers to the surface of the metastable zirconia particles are to treat the zirconia surface with e.g. a silane and then react the triazine with a functional group on the silane. E.g.:

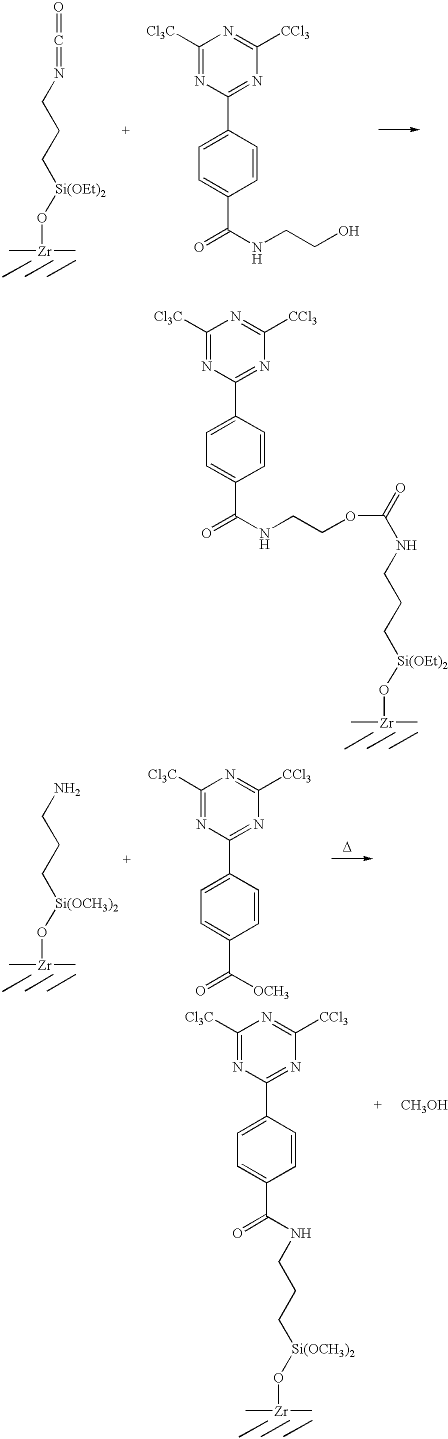

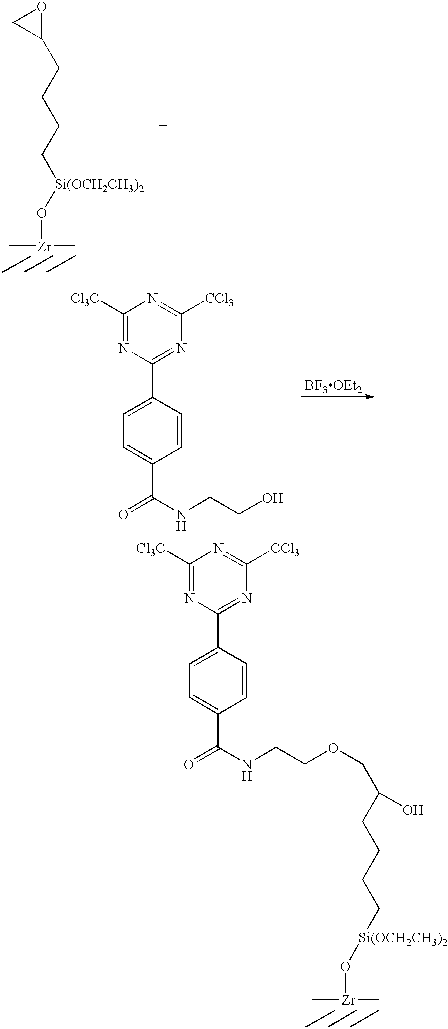

Q. Light Induced Acid Onium Salts

A large number of known compounds and mixtures are suitable for use as radiation-sensitive components which on irradiation form or eliminate preferentially strong acids, such as diazonium, phosphonium, sulfonium and iodonium salts, o-quinonediazide sulfochlorides combinations. It appears probable that acid (even superacids) are produced from these radiation-sensitive components, the reaction can be described by the following.

Ar₂I⁺X⁻→ArI⁺\dot{}X⁻+A\dot{r}

ArI⁺\dot{}X⁻+RH→ArI+R\dot{}+HX

Where X can be number of different anions:

1) X═F, Cl⁻, Br⁻, where the product upon light irradiation will be HF, HCl or HBr, a trigger molecule for the phase conversion of zirconia.

2) The corresponding base of a strong acid (not X═F⁻, Cl⁻, Br⁻) such as AsF₆⁻, SbF₆⁻, PF₆⁻, BF₄⁻ or CF₃ SO₃⁻. The trigger molecule can be produced by having the corresponding base (F⁻, Cl⁻, Br⁻ or OH⁻) of the known trigger molecules in the resin or on the zirconia e.g.

HX+LiCl→HCl+LiX or

HX+LiOH→H₂O+LiX

3) The special case where X═OH⁻, where the product upon light irradiation will be water H₂O, a trigger molecule for the phase conversion of zirconia.

Photolysis of diaryliodonium salts may be photosensitized in the 400-500 nm region by acridine dyes whereas perylene and other polynuclear aromatic hydrocarbons are effective electron-transfer photosensitizers for the triggering of the acid cleavage of triarylsulfonium salts and dialkylphenacylsulfonium salts.

Below is given some examples of the radiation-sensitive components which on irradiation form or eliminate strong acids:

A special case of this is where HX (X═F⁻, Cl⁻, Br⁻) releasing agents can be made by substituting the anion of the below standing compounds with a halogen anion. These molecules release trigger molecules (HF, HCl, HBr) upon exposure to UV-light e.g.: diphenyliodonium chloride

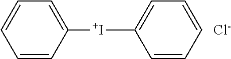

Another special case of this is where water (H₂O) releasing agents can be made by substituting the anion of the below standing compounds with a hydroxyl anion (OH⁻). These molecules release trigger molecules (water) upon exposure to UV-light e.g.: diphenyliodonium hydroxide

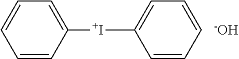

Ref.: U.S. Pat. No. 5,736,296:

(i) Bissulfonyldiazomethanes such as bis(p-toluenesulfonyl)diazomethane, methylsulfonyl-p-toluenesulfonyldiazomethane, 1-cyclohexylsulfonyl-1-(1,1-dimethyl-ethylsulfonyl)diazomethane, bis(1,1-dimethyl-ethylsulfonyl)diazomethane, bis(1-methylethylsulfonyl)diazomethane, bis(cyclohexylsulfonyl)diazomethane, bis(2,4-dimethylphenylsulfonyl)diazomethane, bis-(4-ethylphenylsulfonyl)diazomethane, bis(3-methylphenylsulfonyl)diazomethane, bis(4-methoxyphenylsufonyl)diazomethane, bis(4-fluorophenylsulfonyl)diazomethane, bis(4-chloro-phenylsulfonyl)diazomethane, and bis(4-tert-butylphenylsulfonyl)diazomethane;

(ii) sulfonylcarbonyl alkanes such as 2-methyl-2-(p-toluenesulfonyl)propiophenone, 2-(cyclo-hexyl-carbonyl)-2-(p-toluene sulfonyl)propane, 2-methanesulfonyl-2-methyl-(4-methylthio)proplophenone, and 2,4-dimethyl-2-(p-toluenesulfonyl)pentane -3-one;

(iii) sulfonyl carbonyldiazomethanes such as 1-p-toluenesulfonyl-1-cyclohexylcarbonyldiazomethane, 1-diazo-1-methylsulfonyl-4-phenyl-2-butanone, 1-cyclohexyl-sulfonyl-1-cyclohexylcarbonyldiazomethane, 1-diazo-1-cyclohexylsulfonyl -3,3-di-methyl-2-butanone, 1-diazo-1-(1,1-di-methylethyl sulfonyl)-3,3-di-methyl-2-butanone, 1-acetyl-1-(1-methylethyl sulfonyl)diazomethane, 1-diazo-1-(p-toluenesulfonyl)-3,3-dimethyl-2-butanone, 1-diazo-1-benzenesulfonyl-3,3-di-methyl-2-butanone, 1-diazo-1-(p-toluene sulfonyl)-3-methyl-2-butanone, 2-diazo-2-(p-toluenesulfonyl)cyclohexylacetate, 2-diazo-2-benzene sulfonyl tert-butyl acetate, 2-diazo-2-methanesulfonyl iso-propyl acetate, 2-diazo-2-benzenesulfonyl cyclohexyl acetate, and 2-diazo-2-(p-toluenesulfonyl)tert-butyl acetate;

(iv) nitrobenzyl derivatives such as 2-nitrobenzyl-p-toluenesulfonate, 2,6-dinitrobenzyl-p-toluenesulfonate, and 2,4-dinitrobenzyl-p-trifluoro-methylbenzenesulfonate; and

(v) esters of polyhydroxy compounds and aliphatic or aromatic sulfonic acids such as pyrogallic methane sulfonate ester (pyrogallol trimesylate), pyrogallic benzene sulfonate ester, pyrogallic p-toluene sulfonate ester, pyrogallic p-methoxy benzene sulfonate ester, pyrogallic mesitylene sulfonate ester, pyrogallic benzylsulfonate ester, alkyl gallic acid methane sulfonate ester, alkyl gallic acid benzene sulfonate ester, alkyl gallic acid p-toluene sulfonate ester, alkyl gallic acid p-methoxy benzene sulfonate ester, alkyl gallic acid mesitylene sulfonate ester, and alkyl gallic acid benzylsulfonate ester.

Preferred is the alkyl group in the afore-mentioned alkyl gallic acid where the alkyl group has from 1 to 15 carbon atoms, and especially octyl group or lauryl group. (vi) onium salt-based acid-generating agents to be in a general formula (II) and (III), and (vii) benzoin tosylate-based acid-generating agents to be in a general formula (IV) may be used. A general formula (II);

RI⁺R′X⁻ (II)

where R and R′ are aryl groups or aryl groups having a substituent and may be respectively identical or different; X⁻ is any of AsF₆⁻, SbF₆⁻, PF₆⁻, BF₄⁻, or CF₃ SO₃⁻, F⁻, Cl⁻, Br⁻, OH⁻;

and a general formula (III);

R(R′)S⁺R″X⁻ (III)

where R, R′, and R″ are aryl groups or aryl groups having a substituent and may be respectively identical or different; X⁻ is any of AsF₆⁻, SbF₆⁻, PF₆⁻BF₄⁻, or CF₃ SO₃⁻, F⁻, Cl⁻, Br⁻, OH⁻

A general formula (IV);

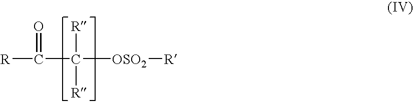

where R and R′ are aryl groups or aryl groups having a substituent and may be identical or different; R″ and R′″ are hydrogen atoms, C₁₋₆-groups, hydroxyl groups, or aryl groups and may be identical or different. n is 0 or 1.

The following are offered as specific onium salts presented by general formulas (II) and (III).

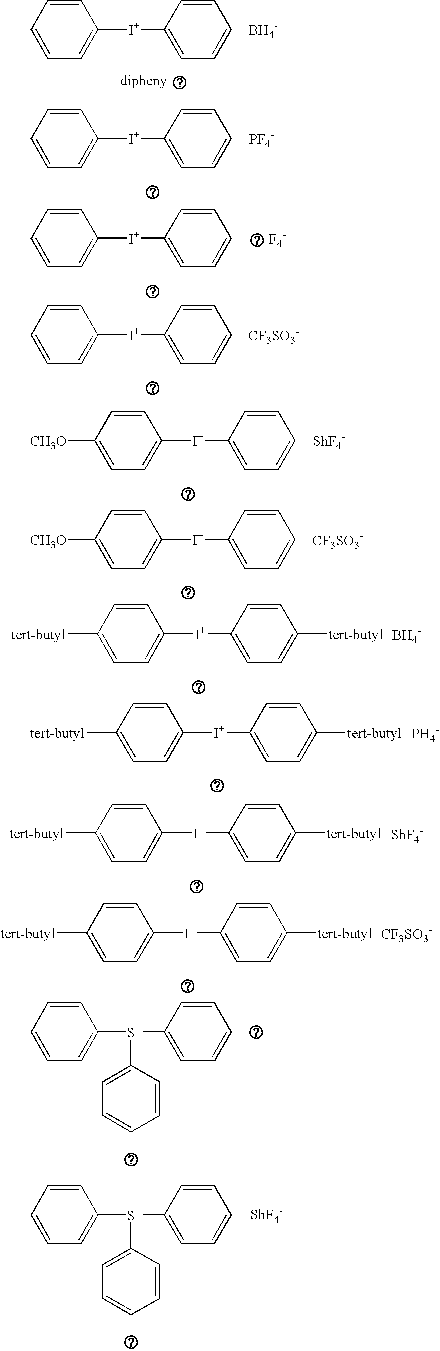

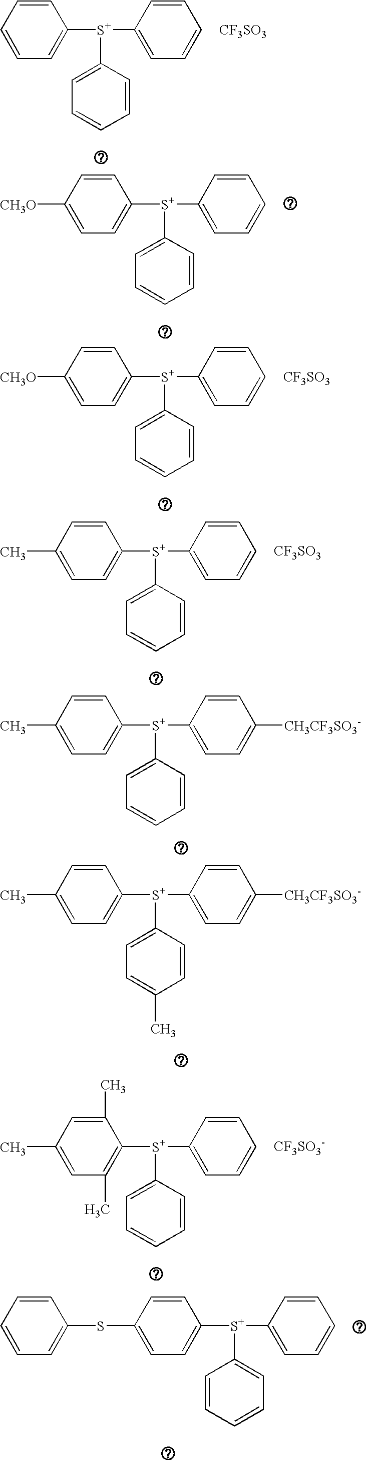

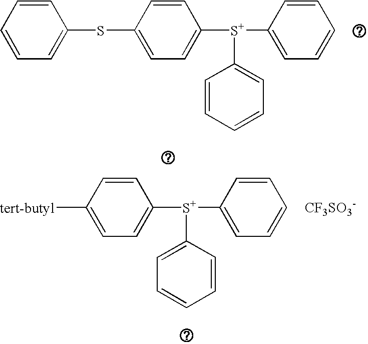

(vii) The following compounds are offered as specific benzoin tosylate-based acid-forming agents.

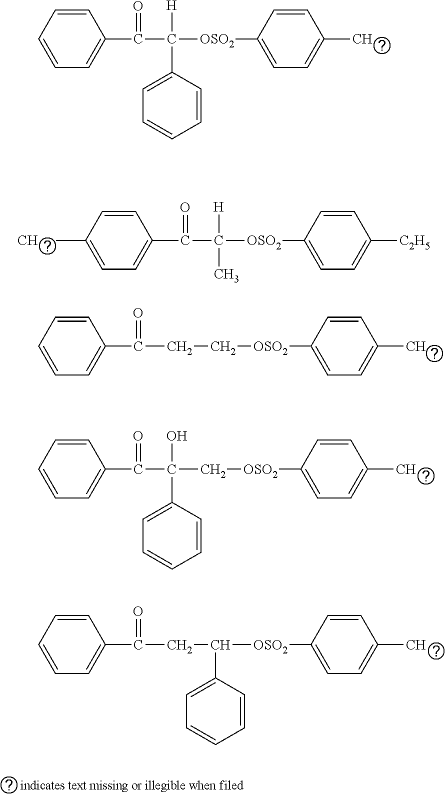

One of these acid-forming agents may be used, or two or more may be used in combination. Particularly, the combination is preferred since the composition containing the mixture has high sensitivity.

As the acid-generating agents to be in the resists for light (blue), other acid-generating agents than the above-mentioned (i) and (iii) can be employed.

Ref.: U.S. Pat. No. 6,770,419 B2:

Onium salts such as triaryl sulfonium or diaryliodonium hexafluoroantimonate, hexafluoroarsenates, triflates, perfluoroalkane sulfonates (e.g., perfluoromethane sulfonate, perfluorobutane, perfluorohexane sulfonate, perfluorooctane sulfonate, etc.), perfluoroalkyl sulfonyl imide, perfluoroalkyl sulfonyl methide, perfluoroaryl sulfonyl imide, perfluoroaryl sulfonyl methide; substituted aryl sulfonates such as pyrogallols (e.g. trimesylate of pyrogallol or tris(sulfonate) of pyrogallol), sulfonate esters of hydroxyimides, N-sulfonyloxynaphthalimides (N-camphorsulfonyloxynaphthalimide, N-pentafluorobenzenesulfonyloxynaphthalimide), (α-α′bis-sulfonyl diazomethanes, naphthoquinone-4-diazides, alkyl disulfones and others. R: Water-Releasing Agent

A better application of the above mentioned water-releasing triggers would be to chemical anchor the triggers to the surface of the metastable zirconia particles. This would ensure that the triggering molecules were close to the reactive sites of zirconia thereby inducing a fast phase-transformation and reducing the risk of other chemical reactions e.g. with the monomer resin. The chemical anchoring could be done with the use of a silane-, phosphate-, carboxylic acid, hydroxamic acid or a carbamate- group and done with surface treatment of the zirconia particles.

Definitions

In the present context, the term “C₁₋₆-alkyl” is intended to mean a linear, cyclic or branched hydrocarbon group having 1 to 6 carbon atoms, such as methyl, ethyl, propyl, iso-propyl, pentyl, cyclopentyl, hexyl, cyclohexyl.

Similarly, the term “C₂₋₆-alkenyl” is intended to cover linear, cyclic or branched hydrocarbon groups having 2 to 6 carbon atoms and comprising one unsaturated bond. Examples of alkenyl groups are vinyl, allyl, butenyl, pentenyl, hexenyl, heptenyl, octenyl, heptadecaenyl. Preferred examples of alkenyl are vinyl, allyl, butenyl, especially allyl.

In the present context, i.e. in connection with the terms “alkyl”, “alkenyl” and the like, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxy, aryl-amino, arylcarbonyl, aryloxycarbonyl, arylcarbonyloxy, arylaminocarbonyl, arylcarbonyl-amino, heteroaryl, heteroaryloxy, heteroarylamino, heteroarylcarbonyl, heteroaryloxy-carbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxy-carbonyl, heterocyclylcarbonyloxy, heterocyclylaminocarbonyl, heterocyclylcarbonylamino, amino, mono- and di(C₁₋₆-alkyl)amino, —N(C₁₋₄-alkyl)₃⁺, carbamoyl, mono- and di(C₁₋₆-alkyl)-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

Typically, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl, heteroaryl and heterocyclyl may be substituted as specifically described below for aryl, heteroaryl and heterocyclyl.

In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen.

The terms “halogen” and “halo” include fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The terms “heteroaryl” and “heteroaromatic” are intended to refer to a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexahydro-pyrimidine, diazepane, diazocane, pyrrolidine, piperidine, azepane, azocane, aziridine, azirine, azetidine, pyroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “heteroaryl”, “heterocyclyl” and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), carboxy, C₁₋₆-alkoxycarbonyl, C₁₋₆-alkylcarbonyl, formyl, aryl, aryloxy, arylamino, aryloxy-carbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbony-lamino, halogen, C₁₋₆-alkylthio, C₁₋₆-alkyl-sulphonyl-amino, or guanidino.

Typically, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Moreover, it should be understood that the compounds may be present as enantiomers or diastereomers. The present invention encompasses each and every of such possible enantiomers and diastereomers as well as racemates and mixtures enriched with respect to one or the possible enantiomers or diastereomers.

Filler/Filler Ingredient

In view of the above, it is apparent that the one or more fillers, and in particular the one or more filler ingredients and the nanofillers, are important constituents of the composite material.

Fillers are frequently used in connection with polymeric materials in order to provide desirable mechanical properties of such materials, e.g. abrasion resistance, opacity, colour, radiopacity, hardness, compressive strength, compressive modulus, flexural strength, flexural modulus, etc.

Such fillers may be selected from one or more of a wide variety of materials, e.g. those that are suitable for the use in dental and/or orthodontic composite materials.

Fillers can be inorganic materials or cross-linked organic materials that are insoluble in the resin component of the composition. Cross-linked organic materials may as such be filled with an inorganic filler. The filler should—in particular for dental uses—be nontoxic and suitable for use in the mouth. The filler can be radiopaque or radiolucent. The filler typically is substantially insoluble in water.

The term “filler” is to be understood in the normal sense, and fillers conventionally used in composite materials in combination with polymer are also useful in the present context. The polymerizable resin base (see further below) can be said to constitute the “continuous” phase wherein the filler is dispersed.

Some examples of suitable inorganic fillers are naturally occurring or synthetic materials including, but not limited to: quartz; nitrides (e.g. silicon nitride); glasses derived from, for example, Zr, Sr, Ce, Sb, Sn, Ba, Zn, and Al; feldspar; borosilicate glass; kaolin; talc; titania; low Mohs hardness fillers such as those described in U.S. Pat. No. 4,695,251 (Randklev); and silica particles (e.g. submicron pyrogenic silicas such as those available under the trade designations AEROSIL, including “OX 50,” “130,” “150” and “200” silicas from Degussa AG, Hanau, Germany and CAB-O-SIL M5 silica from Cabot Corp., Tuscola, Ill.). Examples of suitable organic filler particles include filled or unfilled pulverized polycarbonates, polyepoxides, and the like.

Other illustrative examples of fillers are barium sulfate (BaSO₄), calcium carbonate (CaCO₃), magnesium hydroxide (Mg(OH)₂), quartz (SiO₂), titanium dioxide (TiO2), zirconia (ZrO₂), alumina (Al₂O₃), lantania (La₂O₃), amorphous silica, silica-zirconia, silica-titania, barium oxide (BaO), barium magnesium aluminosilicate glass, barium aluminoborosilicate glass (BAG), barium-, strontium- or zirconium-containing glass, milled glass, fine YF₃ or YbF₅ particles, glass fibres, metal alloys, etc. Metal oxides, e.g. titanium dioxide (TiO₂) and zirconia (ZrO₂), alumina (Al₂O₃), lantania (La₂O₃), constitute a particularly useful group of fillers for use in the composite materials of the present invention.

In one interesting embodiment, at least 5%, e.g. at least 10%, or even at least 20%, by weight of the one or more fillers are glass-particles. It is believed that inclusion of glass particles may further improve the optical (and thereby aesthetic) properties of the composite material by making it more transparent.

The weight content of the one or more filler materials in the composite material is typically in the range of 5-95%, or 10-90%, such as 30-95%, such as 40-95%, e.g. 60-95%. It should be understood that a combination of two or more fillers may be desirable, just as the particle size distribution of the filler(s) may be fairly broad in order to allow a dense packing of the filler and thereby facilitate incorporation of a high amount of fillers in the composite material. Typically, composite materials have a distribution of one or more sizes of fine particles plus microfine and/or nano-size filler (5-15%). This distribution permits more efficient packing, whereby the smaller particles fill the spaces between the large particles. This allows for filler content, e.g., as high as 77-87% by weight. An example of a one size distribution filler would be 0.4 μm structural micro-filler, with the distribution as follows: 10% by weight of the filler particles have a mean particle size of less than 0.28 μm; 50% by weight of the filler particles have a mean particle size of less than 0.44 μm; 90% by weight of the filler particles have a mean particle size of less than 0.66 μm.

Typically, the particle size of the filler(s) is in the range of 0.01-50 μm, such as in the range of 0.02-25 μm, and—as mentioned above—include nanofillers having a particle size of at the most 100 nm.

In some embodiments, the particle size of the filler(s) is/are in the range of 0.2-20 μm with some very fine particles of about 0.04 μm. As an example, fairly large filler particles may be used in combination with amorphous silica in order to allow for a dense packing of the fillers.

The term “particle size” is intended to mean the shortest dimension of the particulate material in question. In the event of spherical particles, the diameter is the “particle size”, whereas the width is the “particle size” for a fiber- or needle-shaped particulate material. It should of course be understood that an important feature of such particles is the actual crystal size in that the crystal size (and not the particle size) will be determinative for the preferred crystal phase under given conditions (see also further below).

As used herein the term “nanofiller” is used synonymously with “nanosized particles” and “nanoparticles” and refers to filler particles having a size of at the most 100 nm (nanometers). As used herein for a spherical particle, “size” refers to the diameter of the particle. As used herein for a non-spherical particle, “size” refers to the longest dimension of the particle.

This being said, the weight ratio between (i) the nanofillers and (ii) fraction of the one or more fillers not being said nanofillers appears to play a certain role, and is typically in the range of 10:90 to 100:0, preferably 10:90 to 40:60, in particular 10:90 to 30:70.

In the embodiment where the composite material is for dental use, particularly useful fillers are zirconia, amorphous silica, milled barium-, strontium- or zirconium-containing glass, milled acid-etchable glass, fine YF₃ or YbF₅ particles, glass fibres, etc.

The one or more fillers comprise at least one filler ingredient. The term “filler ingredient” is intended to mean the filler or a fraction of the filler having particular physical properties, namely the inherent ability to compensate (by expansion) for volumetric shrinkage caused by polymerization and curing of the resin base. Thus, a certain filler, e.g. zirconia, may be included in the composite material, and a certain fraction of these filler particles may have particular physical properties, i.e. exist in a metastable crystalline phase (see the following), and thereby constitute the filler ingredient.

The particle size of the filler ingredient(s) is/are typically in the range of 0.01-50 μm. The filler ingredient(s) typically constitute(s) 20-100% of the total weight of the one or more fillers, e.g. 30-100%, such as 40-100% or 50-100%.

When calculated on the basis of the total weight of the composite material, the filler ingredient(s) typically constitute(s) 15-95% of the total weight of the composite material, e.g. 25-95%, such as 30-95%, more specifically 60-95%.

The one or more filler ingredients are present in a metastable first phase and are able to undergo a martensitic transformation to a stable second phase, where the volume ratio between said stable second phase and said metastable first phase of said filler ingredient(s) is at least 1.005, such as at least 1.01 or even at least 1.02 or at least 1.03.

In the present context, the term “metastable first phase” means that the filler ingredient existing in such as phase has a free energy that is higher than the free energy of the second phase, and that an activation barrier (F*) must be overcome before transformation from the first phase (high energy state) to the second phase (low energy state) can proceed. Thus, the phase transformation does not proceed spontaneously. It should be understood that the “system” in which the filler ingredient is metastable is the composite material including all its constituents, i.e. the composite material before curing.

The phase transformation is martensitic, which by definition means that the crystal structure of the filler ingredient needs no extra atoms to undergo the transformation. Thus, the transformation can be very fast, almost instantaneous.

The expression “free energy” refers to the sum of free energies from the particle bulk, the particle surface and strain contributions. For most practical purposes, only the free energies from the particle bulk and the particle surface need to be considered.

Thus, when considering various materials as potential filler ingredients, it is relevant to take into consideration the three main requirements:

1. A first requirement for the filler ingredient is that the second crystalline phase thereof, within the selected particle size range, is “stable” under “standard” conditions, i.e. standard pressure (101.3 kPa) and at least one temperature in the range of 10-50° C., i.e. corresponding to the conditions under which the product is used.

2. A second requirement for the filler ingredient is that a metastable first crystalline phase of the filler ingredient can exist under the same “standard” conditions.

3. A third requirement for the filler ingredient is that the specific volume ratio between said stable second phase and said metastable first phase of said filler ingredient(s) is at least 1.005.

The expression “stable” refers to a phase which does not transform spontaneously under the conditions required for transforming the filler ingredient from the first metastable phase. Thus, the “stable” phase need not always be the phase with the “globally” lowest free energy, but it often will be.

The filler ingredients relevant in the present context comprise particular crystalline forms of some of the fillers mentioned above, in particular of the metal oxide fillers. A very useful example hereof is ZrO₂ (see in particular the section “Populations of zirconia particles” further below). Zirconia can exist in three major crystalline phases: the tetragonal phase, the cubic phase and the monoclinic phase. The specific volume (density⁻¹) of the three phases is 0.16, 0.16 and 0.17 cm³/g, respectively. Thus, the monoclinic (the second phase) and one of the former two phases (the first phase) have a volume ratio higher than 1.005 (i.e. 1.045 and 1.046, respectively). The tetragonal and the cubic phases have higher bulk energy than the monoclinic phase at the standard conditions.

Illustrative examples of filler ingredients are:

Zirconia in the metastable tetragonal phase (specific volume=0.16 cm³/g) which can transform into the monoclinic phase (specific volume=0.17 cm³/g) (volume ratio=1.045);

Zirconia in the metastable cubic phase (specific volume=0.16 cm³/g) which can transform into the monoclinic phase (specific volume=0.17 cm³/g) (volume ratio=1.046);

Lanthanide sesquioxides (Ln₂O₃), where Ln=Sm to Dy. Transforms from monoclinic to cubic phase at 600-2200° C. with a volume expansion of 10%.

Nickel sulfide (NIS). Transforms from rhombohedral to hexagonal phase at 379° C. with a volume expansion of 4%. Density 5.34 g/mi.

Dicalcium silicate (belite) (Ca₂SiO₄). Transforms from monoclinic to orthorhombic phase at 490° C. with a volume expansion of 12%. Density 3.28 g/ml.

Lutetium borate (LuBO₃). Transforms from hexagonal to rhomhedral phase at 1310° C. with a volume expansion of 8%.

The surface energy of the tetragonal phase of zirconia is lower than the one of the monoclinic phase at standard temperature and pressure, which results in stable tetragonal (pure) zirconia crystals at room temperature. The crystals must be small (<10 nm) for the difference of surface energy to compete with difference of in bulk energy of the tetragonal and monoclinic phase.

For zirconia in the metastable tetragonal or cubic crystalline phase, the particle size is preferably in the range of 5-80,000 nm, such as 20-2000 nm, though it is believed that a mean particle size in the range of 50-1000 nm, such as 50-500 nm, provides the best balance between optical and structural properties.

In one embodiment, the filler ingredient(s) is/are able to undergo the martensitic transformation under the influence of ultrasound.

In view of the above, the filler ingredient(s) preferably include(s) zirconia (ZrO₂) in metastable tetragonal or cubic crystalline phase (see in particular the section “Populations of zirconia particles” further below).

In another embodiment, the filler ingredient(s) is/are able to undergo the martensitic transformation upon exposure to a chemical trigger.

In some instances, the activation barrier (F*) is not sufficiently large to prevent premature transformation from the first phase to the second phase. This may result in a spontaneous transformation upon storage of the composite material. Thus, in some embodiments, it is advantageous to stabilize the native filler ingredient in order to obtain a metastable phase that will not undergo more or less spontaneous, i.e. premature, transformation upon storage of the composite material. Stabilization of the metastable phase can, e.g., be achieved by doping, by surface modification of the filler particles, etc. as will be explained in the following.

In one variant, at least 50% of the nanofillers are zirconia particles.

Doping

Many crystal phases can be stabilized using doping materials. Generally, with increasing amounts of dopant, the more the phase is stabilised. In energy-terms, the activation barrier (F*) becomes higher the more dopant used. In order to trigger the phase transformation, the activation barrier must, however, be low enough for the trigger method to overcome the activation barrier, but high enough so that the transformation does not occur spontaneously.

Zirconia is typically stabilized using up to 20 mol-% of one or more dopants. Zirconla can be stabilized with stabilizer such as calcium, cerium, barium, yttrium, magnesium, aluminum, lanthanum, caesium, gadolinium and the like, as well as oxides and combinations thereof. More specifically, the recommended mol-% content for some useful dopants (if it is decided to include a dopant) is: Y₂O₃ (1-8%), MgO (1-10%), CaO (1-18%), CeO₂ (1-12%), and Sc₂O₂ (1-10%). A dopant level of, e.g., Y₂O₃ of 0-1% will typically not sufficiently stabilize the tetragonal phase and the cubic phase of zirconia, and such doped zirconia will, therefore, still undergo a phase transformation spontaneously to the monoclinic phase at room temperature. Adding too high a level of Y₂O₃, e.g. 8% or more, will stabilise the tetragonal phase and the cubic phase to such an extent that the activation barrier will become too high to overcome with most trigger process. At some point in between the activation barrier, the transformation can be triggered as described below. Adding more dopant will make the triggering more difficult and thus slower. Adding less dopant could make the zirconia unstable and not useful as a filler ingredient. [It should be noted that commercial grade zirconia contains a small fraction of hafnium. Such small amounts of hafnium are neglected in the discussion above, because hafnium is viewed as an integral part of zirconia.]

In a preferred embodiment, the metastable phase of the zirconia is stabilized by doping with an oxide selected from Y₂O₃, MgO, CaO, CeO₂, and Sc₂O₃.

Depending on the activation energy as explained--above, the levels of dopants for ZrO₂ could be Y₂O₃ (1-5%), MgO (1-5%), CaO (1-10%), and CeO₂ (1-6%), but for ideal zirconia crystal doping is not necessary so more specifically about 0-2%.

Surface Modification

Surface energy can be changed by surface modification. By modification of the surface by adsorption of a chemical constituent, it is possible to lower the surface energy of the first phase so that the sum of the surface energy and the bulk energy becomes lower than the surface energy and the bulk energy for the second phase, and thereby “reverse” the stability order of the first and second phase. In this way, the “metastability” of the first phase arises because the first phase is only “stable” as long as the chemical constituent is adsorbed thereto. Thus, the first phase is stabilised until the surface modification is altered or removed, e.g. by treatment with a chemical trigger.

Generally, the surface of the filler particles (nanofillers, filler ingredients, etc.) can also be treated with a coupling agent in order to enhance the bond between the filler and the resin. Suitable coupling agents include gamma-methacryloxypropyltrimethoxysilane, gamma-mercaptopropyltriethoxysilane, gamma-aminopropyltrimethoxysilane, and the like. Examples of useful silane coupling agents are those available from GE silicones, as SILQUEST A-174 and SILQUEST A-1230. For some embodiments of the composite material, the composite materials may include at least 1% by weight, more preferably at least 2% by weight, and most preferably at least 5% by weight other filler, based on the total weight of the composite material. For such embodiments, composite materials of the present invention preferably include at most 40% by weight, more preferably at most 20% by weight, and most preferably at most 15% by weight other filler, based on the total weight of the composite material.

Polymerizable Resin Base

Another important constituent of the composite material is the polymerizable resin base.

The term “polymerizable resin base” is intended to mean a composition of a constituent or a mixture of constituents such as monomer, dimers, oligomers, prepolymers, etc. that can undergo polymerization so as to form a polymer or polymer network. By polymer is typically meant an organic polymer. The resin base is typically classified according to the major monomer constituents.

The weight content of the polymerizable resin base in the composite material is typically in the range of 5-95%, or 5-90%, e.g. 5-70%, such as 5-60%, e.g. 5-40%.

Virtually any polymerizable resin base can be used within the present context. Polymerizable resin bases of particular interest are, of course, such that upon curing will cause a volumetric shrinkage of the composite material when used without a compensating filler ingredient.

The term “curing” is intended to mean the polymerisation and hardening of the resin base.

One class of preferred hardenable resins are materials having free radically active functional groups and include monomers, oligomers, and polymers having one or more ethylenically unsaturated groups. Alternatively, the hardenable resin can be a material from the class of resins that include cationically active functional groups. In another alternative, a mixture of hardenable resins that include both cationically curable and free radically curable resins may be used for the dental materials of the invention.

In the class of hardenable resins having free radically active functional groups, suitable materials for use in the invention contain at least one ethylenically unsaturated bond, and are capable of undergoing addition polymerization. Such free radically polymerizable materials include mono-, di- or poly- acrylates and methacrylates such as methyl acrylate, methyl methacrylate, ethyl acrylate, isopropyl methacrylate, n-hexyl acrylate, stearyl acrylate, allyl acrylate, glycerol diacrylate, glycerol triacrylate, ethyleneglycol diacrylate, diethyleneglycol diacrylate, triethyleneglycol dimethacrylate, 1,3-propanediol diacrylate, 1,3-propanediol dimethacrylate, trimethylolpropane triacrylate, 1,2,4-butanetriol trimethacrylate, 1,4-cyclohexanediol diacrylate, pentaerythritol triacrylate, pentaerythritol tetraacrylate, pentaerythritol tetramethacrylate, sorbitol hexacrylate, the diglycidyl methacrylate of bis-phenol A (“Bis-GMA”), bis[1-(2-acryloxy)]-p-ethoxyphenyldimethylmethane, bis[1-(3-acryloxy-2-hydroxy)]-p-propoxyphenyldimethylmethane, and trishydroxyethyl-isocyanurate trimethacrylate; the bis-acrylates and bis-methacrylates of polyethylene glycols of molecular weight 200-500, copolymerizable mixtures of acrylated monomers such as those in U.S. Pat. No. 4,652,274, and acrylated oligomers such as those of U.S. Pat. No. 4,642,126; and vinyl compounds such as styrene, diallyl phthalate, divinyl succinate, divinyladipate and divinylphthalate. Mixtures of two or more of these free radically polymerizable materials can be used if desired.

An alternative class of hardenable resins useful in the dental materials of the invention may include cationically active functional groups. Materials having cationically active functional groups include cationically polymerizable epoxy resins, vinyl ethers, oxetanes, spiro-orthocarbonates, spiro-orthoesters, and the like.

Preferred materials having cationically active functional groups are epoxy resins. Such materials are organic compounds having an oxirane ring which is polymerizable by ring opening. These materials include monomeric epoxy compounds and epoxides of the polymeric type and can be aliphatic, cycloaliphatic, aromatic or heterocyclic. These materials generally have, on the average, at least 1 polymerizable epoxy group per molecule, preferably at least about 1.5 and more preferably at least about 2 polymerizable epoxy groups per molecule. The polymeric epoxides include linear polymers having terminal epoxy groups (e.g. a diglycidyl ether of a polyoxyalkylene glycol), polymers having skeletal oxirane units (e.g. polybutadiene polyepoxide), and polymers having pendent epoxy groups (e.g. a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, nitro groups, phosphate groups, and the like. The molecular weight of the epoxy-containing materials may vary from about 58 to about 100,000 or more.

Useful epoxy-containing materials include those which contain cyclohexane oxide groups such as epoxycyclohexanecarboxylates, typified by 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexylmethyl)adipate. For a more detailed list of useful epoxides of this nature, reference is made to the U.S. Pat. No. 3,117,099, which is incorporated herein by reference.

Particularly interesting resin bases that are useful for dental applications are those based on compounds selected from the group consisting of methacrylic acid (MA), methylmethacrylate (MMA), 2-hydroxyethyl-methacrylate (HEMA), triethyleneglycol dimethacrylate (TEGDMA), bisphenol-A-glycidyl dimethacrylate (BisGMA), bisphenol-A-propyl dimethacrylate (BisPMA), urethane-dimethacrylate (UDMA), and HEMA condensed with butanetetracarboxylic acid (TCB), as well as those based on combinations of the above-mentioned compounds. Such resin bases are, e.g., disclosed and discussed in U.S. Pat. No. 6,572,693. A particularly useful combination of compounds is TEGDMA and BisGMA, see, e.g., U.S. Pat. No. 3,066,112.

Other Constituents of the Composite Material

The composite material may comprise other constituents which provide beneficial Theological, cosmetic, etc. properties. Examples of such other constituents are dyes, flavorants polymerisation initiators and co-initiators, stabilizers, fluoride releasing materials, sizing agents, antimicrobial ingredients, fire retardants.

Thus, the resin base may include initiators and co-initiators, and illustrative examples of such compounds, particularly for use in dental applications, are benzoylperoxide (BPO), camphorquinone (CPQ), phenylpropanedione (PPD) and N,N-di(2-hydroxyethyl)-p-toluidine (DEPT), N,N-dimethyl-p-aminobenzoic acid ethyl ester (DAEM).

Shading can be achieved by using a number of color pigments. These include metal oxides, which provide the wide variety of colors of the composite; for example, oxides of iron can act as a yellow, red to brown pigment, copper as a green pigment, titanium as a yellowish-brown pigment, and cobalt imparts a blue color.

Fluorescence is more subtle optical properties that further enhance the natural-looking, life- like appearance or “vitality” of the tooth. Fluorescence is defined as the emission of electromagnetic radiation that is caused by the flow of some form of energy into the emitting body, which ceases abruptly when the excitation ceases. In natural teeth, components of the enamel, including hydroxyapatite, fluoresce under long wavelength ultraviolet light, emitting a white visible light. This phenomenon is subtle in natural daylight but still adds further to the vitality of the tooth. In contrast, under certain lighting conditions, the lack of fluorescence in a restorative material may become alarming. Under “black light” conditions, such as that often used in discotheque-type night clubs, if a restoration does not fluoresce, the contrast between the tooth and restoration may be so great that the tooth may actually appear to be missing. Fluorescence can, e.g., be achieved by adding an anthracene-like molecule.

The weight content of other constituents in the composite material is typically in the range of 0-10%, such as 0-5%, e.g. 0-4% or 1-5%.

Dental Filling Materials

In view of the above, the present invention also provides a dental filling material in the form of a composite material as defined above. In particular, the filler ingredient(s) of the composite material include(s) zirconia (ZrO₂) in metastable tetragonal or cubic crystalline phase.

In a particularly interesting embodiment, the dental filling material consists of:

40-90% (e.g. 40-85% ) by weight of the one or more fillers, wherein said one or more fillers comprise at least one filler ingredient, said filler ingredient(s) include(s) metastable zirconia in the tetragonal or cubic crystalline phase, and wherein said material further comprises one or more water- or acid-releasing agents;

10-60% (e.g. 15-60%) by weight of the a polymerizable resin base, said resin base being based on one or more compound selected from the group consisting of methacrylic acid (MA), methylmethacrylate (MMA), 2-hydroxyethyl-methacrylate (HEMA), triethyleneglycol dimethacrylate (TEGDMA), bisphenol-A-glycidyl dimethacrylate (BisGMA), bisphenol-A-propyl dimethacrylate (BisPMA), urethane-dimethacrylate (UDMA), and HEMA condensed with butanetetracarboxylic acid (TCB);

0-5% by weight of additives; and

0-4% by weight of solvents and/or water.

In order to avoid premature curing of the polymerizable resin base, it may be advantageous to prepare and store the composite material as a two-component material intended for mixing immediately prior to use.

Use of the Composite Materials

The composite materials may be used and are cured essentially as conventional composite materials of the same type, except for the fact that the martensitic transformation should be controlled along with the curing of the resin base, i.e. at least in part by the chemical trigger(s) resulting from the water- and/or acid-releasing agents.

Generally, it is believed that the martensitic transformation can be activated either by physical means (e.g. application of mechanical pressure, tension, ultrasound, Roentgen irradiation, microwaves, longitudinal waves, electromagnetic irradiation such as light, near infrared irradiation, heating, etc.) or by chemical means (e.g. modification of the surface free energy by contacting the surface of the filler ingredient particles with a chemical, e.g. a constituent of the composite material or an additive such as water). Hence, it should be understood that the martensitic transformation may be further triggered by such means, although it is believed that the water- and/or acid-releasing agents will contribute significantly, or even completely, to the triggering of the martensitic transformation of the filler ingredient(s).

It should be understood that the martensitic transformation of the filler ingredient preferably shall take place with the curing (polymerization and hardening) of the resin base. However, since the crystals are small, the expansion due to phase transformation will not cause deterioration of the mechanical properties of the cured compound. Therefore, transformation triggered by slow mechanisms, e.g., diffusion of water into the cured compound or inner tensile stress build up by shrinkage from curing, will happen after the curing. Triggering the transformation before the curing is undesired since the volume compensating effect will be less or lost depending on how much is transformed before curing is initiated.

In order to make a phase transformation of a system where the first phase is metastable, but where the activation barrier is high because of a low surface energy of the first phase, the activation barrier can be lowered by surface modification. The activation of the phase transformation can be initiated by surface modification. The activation barrier will be the energy needed to make a surface modification that makes the surface energy of the phase higher (or make it more similar to the surface of the second phase).

Method of the Invention

In view of the above, the present invention also provides a method of controlling the volumetric shrinkage of a composite material upon curing, comprising the step of:

(a) providing a composite material comprising one or more fillers and a polymerizable resin base, wherein said one or more fillers comprise at least one filler ingredient, said filler ingredient(s) being present in a metastable first phase and being able to undergo a martensitic transformation to a stable second phase, the volume ratio between said stable second phase and said metastable first phase of said filler ingredient(s) being at least 1.005, and wherein said material further comprises one or more water- or acid-releasing agents;

(b) allowing the resin base to polymerize and cure, and allowing the filler ingredient(s) to undergo a martensitic transformation from said first metastable phase to said second stable phase.

Preferably, the filler ingredient(s) should be triggered to undergo the martensitic transformation either simultaneous with the curing or subsequent to the curing in order to fully benefit from the volumetric expansion of the filler ingredient(s).

In another embodiment, the martensitic transformation of the filler ingredient(s) is initiated by exposure of the surface of the filler ingredient(s) to a chemical trigger. In this instance, the martensitic transformation is preferably triggered simultaneously with or after the curing is initiated, but before the curing is completed.

More specifically, the present invention further provides a method of reconstructing a tooth, comprising the step of

(a) preparing a cavity in the tooth;

(b) filing said cavity with a dental filling material as defined above; and

(c) allowing the resin base of the dental filling material to polymerize and cure, and allowing the filler ingredient(s) of the dental filling material to undergo a martensitic transformation from a first metastable phase to a second stable phase.

The above-defined method for the reconstruction of a tooth may generally comprise further steps obvious to the person skilled in the art of dentistry.

In one embodiment, the martensitic transformation of the filler ingredient(s) is initiated by application of ultrasound (10-1000 kHz). In another embodiment, the martensitic transformation of the filler ingredient(s) is initiated by exposure of the surface of the filler ingredient(s) to a chemical trigger.

In a currently highly preferred embodiment of the above described methods, the water- or acid-releasing agent(s) comprise(s) at least one triazine compound, said triazine compound comprising one or two trihalomethyl groups represented by the following general formula (I):

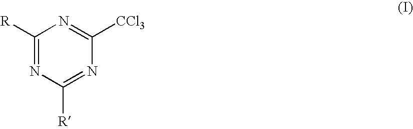

wherein CCl₃ may be replaced by a CF₃ group; R represents the attachment point for an organic moiety; and R′ is selected from the group consisting of a hydrogen atom, a further trihalomethyl group, a substituted C₁₋₆-alkyl group, an unsubstituted C₁₋₆-alkyl group, a substituted aryl group, an unsubstituted aryl group, and a substituted C₂₋₆-alkenyl group.

More generally, the present invention also relates to a composite material as defined herein for use in medicine, in particular in dentistry.

The present invention also relates to the use of a filler ingredient for the preparation of a composite material for reconstructing a tooth in a mammal, said filler ingredient having a metastable first phase and being able to undergo a martensitic transformation to a stable second phase, the volume ratio between said stable second phase and said metastable first phase of said filler ingredient being at least 1.005, and wherein said material further comprises one or more water- or acid-releasing agents. The nanofillers, filler ingredient(s) and the composite material are preferably as defined herein.

A Population of Zirconia Particles

It has been found that metastable zirconia may be used as a particularly suitable filler in composite materials. In particular, it has been found that zirconia which is capable of allowing a martensitic transformation to a stable second phase is particularly useful in order to counter the shrinkage normally occurring in composite materials.

Thus, a further aspect of the present invention relates to a population of zirconia particles having an average particle size in the range of 50-2000 nm, said particles being present in a metastable first phase and being able to undergo a martensitic transformation to a stable second phase, said transformation being effected to an extent of at least 80% within 300 sec when tested in the “Zirconia Particle Transformation Test” defined herein.

Furthermore, the present invention also relates to method for preparing such populations of zirconia particles.

The zirconia particles of the above-defined populations are present in a metastable first phase and are able to undergo a martensitic transformation to a stable second phase. Preferably, the volume ratio between said stable second phase and said metastable first phase of said zirconia particles is at least 1.005, such as at least 1.01 or even at least 1.02 or at least 1.03.

As mentioned above, the particles of the population of the first aspect of the invention are present in a metastable first phase and being able to undergo a martensitic transformation to a stable second phase, said transformation being effected to an extent of at least 80% within 300 sec when tested in the “Zirconia Particle Transformation Test” defined herein. Preferably, the transformation is effected to an extent of at 80% within 10-100 sec, such as within 20-60 sec.

Thus, when considering various crystal forms and particle sizes of the zirconia particles, it is relevant to take into consideration the two main requirements:

1. A first requirement for the zirconia particles is that the second crystalline phase thereof, within the selected particle size range, is “stable” under “standard” conditions, i.e. standard pressure (101.3 kPa) and at least one temperature in the range of 10-50° C., i.e. corresponding to the conditions under which the product (typically a composite material) is used.

2. A second requirement for the zirconia particles is that a metastable first crystalline phase of the zirconia particles can exist the under the same “standard” conditions.

For zirconia in the metastable tetragonal or cubic crystalline phase, the particle size is preferably in the range of 50-2000 nm, though it is believed that a mean particle size in the range of 50-1000 nm provides the best balance between optical and structural properties.

The zirconia particles are able to undergo the martensitic transformation under the influence of ultrasound. The zirconia particles may also undergo the martensitic transformation upon exposure to a chemical trigger.

In view of the above, the filler ingredient(s) preferably include(s) zirconia (ZrO₂) in metastable tetragonal or cubic crystalline phase.

Stabilization of the metastable phase can, e.g., be achieved by doping, by surface modification of the zirconia, etc. as it is explained hereinabove.

### Embodiments

In order to obtain zirconia particles that could undergo a fast phase transformation, a large surface area, e.g. 10-250 m²/g or even better 50-200 m²/g, of the particles is preferred and also obtainable by the means described herein.

Thus, a further aspect of the present invention relates to a population of zirconia particles having an average particle size in the range of 50-2000 nm and a BET surface area of in the range of 10-250 m²/g, said particles being present in a metastable first phase and being able to undergo a martensitic transformation to a stable second phase.

Preferably, this population of zirconia particles allows for a martensitic transformation to be effected to an extent of at least 80% within 300 sec when tested in the “Zirconia Particle Transformation Test” defined herein.

As mentioned above, the average particle size is typically in the range of 50-2000 nm, such as in the range of 50-1000 nm, in particular 100-600 nm.

Although the particles size of the zirconia particles generally is in the range of 50-2000 nm, it is believed that the particles may comprise smaller crystal domains with a homogeneous crystal lattice. Accordingly, it is preferred that the particles have crystal domain sizes in the range of 1-100 nm, such as in the range of 4-50 nm, such as 5-9 nm.

Furthermore, it is believed that the zirconia particles advantageously may have a certain porosity in order to allow for a rapid transformation (as described herein). Thus, the average pore size of the particles is preferably in the range of 10-50 nm.

With respect to the porosity, it is believed that zirconia particles having a porosity in the range of 0.1-20%, such as 0.2-10%, are particularly interesting.

Particularly interesting populations are those where the zirconia particles have
a. an average particle size in the range of 50-2000 nm and a BET surface area of in the range of 10-250 m²/g, or
b. an average particle size in the range of 50-1000 nm and a BET surface area of in the range of 10-250 m²/g, or
c. an average particle size in the range of 100-600 nm and a BET surface area of in the range of 10-250 m²/g, or
d. an average particle size in the range of 50-2000 nm and a BET surface area of in the range of 50-200 m²/g, or
e. an average particle size in the range of 50-1000 nm and a BET surface area of In the range of 50-200 m²/g, or
f. an average particle size in the range of 100-600 nm and a BET surface area of in the range of 50-200 m²/g, or
g. an average particle size in the range of 50-2000 nm and a BET surface area of in the range of 50-80 m²/g, or
h. an average particle size in the range of 50-1000 nm and a BET surface area of in the range of 50-80 m²/g, or
i. an average particle size in the range of 100-600 nm and a BET surface area of In the range of 50-80 m²/g, or
j. an average particle size in the range of 50-2000 nm and a BET surface area of in the range of 75-150 m²/g, or
k. an average particle size in the range of 50-1000 nm and a BET surface area of in the range of 75-150 m²/g, or
l. an average particle size in the range of 100-600 nm and a BET surface area of in the range of 75-150 m²/g, or
m. an average particle size in the range of 50-2000 nm and a BET surface area of in the range of 125-200 m²/g, or
n. an average particle size in the range of 50-1000 nm and a BET surface area of in the range of 125-200 m²/g, or
o. an average particle size in the range of 100-600 nm and a BET surface area of in the range of 125-200 m²/g, or
p. an average particle size in the range of 100-350 nm and a BET surface area of in the range of 50-80 m²/g, or
q. an average particle size in the range of 250-500 nm and a BET surface area of in the range of 50-80 m²/g, or
r. an average particle size in the range of 400-600 nm and a BET surface area of in the range of 50-80 m²/g, or
s. an average particle size in the range of 100-350 nm and a BET surface area of in the range of 75-150 m²/g, or
t. an average particle size in the range of 250-500 nm and a BET surface area of in the range of 75-150 m²/g, or
u. an average particle size in the range of 400-600 nm and a BET surface area of in the range of 75-150 m²/g, or
v. an average particle size in the range of 100-350 nm and a BET surface area of in the range of 125-200 m²/g, or
w. an average particle size in the range of 250-500 nm and a BET surface area of in the range of 125-200 m²/g, or
x. an average particle size in the range of 400-600 nm and a BET surface area of In the range of 125-200 m²/g.

Preparation of a Population of Zirconia Particles

The populations of particles defined above may be prepared by one of the methods described in the following.

Method A

One method for the preparation of a population of the above-defined zirconia particles involves heating of amorphous zirconia within a narrow temperature range. Thus, the present invention provides a method for the preparation of a population of zirconia particles as defined hereinabove, said method comprising the step of heating a sample of amorphous zirconia to a temperature within the crystal formation temperature and not higher than the transition temperature of the zirconia from tetragonal to monoclinic both can determined by DSC or XRD. Heating a sample to a temperature that is below the crystal formation temperature will lead to a sample with few or none crystals with no possibility of phase transformation. Heating a sample to a temperature that is much higher (e.g. 200° C. higher) than the crystal formation will gradually turn the sample from the tetragonal phase to a monoclinic phase. However this may be preferably to have heated to a temperature somewhat (say 20° C.) higher than the crystal formation temperature. This ensures that the zirconia is transformed from the amorphous state into the tetragonal phase.

The heating process can be done in normal air standard pressure, but preferably in dry air because humidity (water) promotes the monoclinic phase of zirconia. A dry air flow is therefore preferably, other dry inert atmospheres such as nitrogen, argon or helium could also be used. Since a controlled heating is necessary in order not to create overshoot depending on the oven a heating ramp of 5° C. is useful. Once reached-the set-point temperature the sample should be kept at that temperature long (say 30-120 min) enough to enable the crystallisation process to occur, but not to long (say 24 hours) since sintering of the crystals could create too much of the monoclinic phase.

Preferably, the amorphous zirconia particles have a BET surface area of in the range of 250-550 m²/g, or 250-500 m²/g, such as in the range of 350-500 m²/g.

Such amorphous zirconia may be synthesized from a zirconate, e.g. ZrOCl₂.8H₂O, by precipitation with a basic solution, e.g. a NH₃ solution. After precipitation and filtration, the zirconia is preferably digested at 100° C. in deionised water for a suitably period of time, e.g. in the range of ½-48 hours, such as in the range of 6-12 hours. Alternatively, the amorphous zirconia is synthesized from a zirconate, e.g. ZrOCl₂.8H₂O, by precipitation with a basic solution at pH 10, e.g. a conc. NH₃ solution. After precipitation, the zirconia is preferably digested under reflux (at 100° C.) in the mother liquid for a suitably period of time, e.g. In the range of 6-24 hours, such as in the range of 8-20 hours.

Method B

Another method for the preparation of a population of the above-defined zirconia particles involves the step forming a suspension of a powder of small tetragonal crystals of zirconia in a strong aqueous base e.g. alkali base such as KOH or NaOH under reflux for 24 h. The crystals are then grown in a strong base suspension (1-5 M) to a size, where the bulk energy of the crystals becomes comparable to the surface energy stabilising the tetragonal phase, thus, lowering the activation barrier. The crystals are grown under hydrothermal conditions e.g. high temperatures in the range of 150-200° C. using a closed reactor (an autoclave, pressure reactor) only with use of waters vapour pressure (because of the heating) creating pressures up to 20 bars. Under these conditions a resolvation and reprecipitation takes place. To achieve large enough crystals the zirconia particles must remain in the pressure reactor for 24 h.

Preferably, the suspension is heated for a period of not less than 2 hours.

Composite Materials

Generally, the populations of particles defined above are believed to be particularly useful as filler ingredients-in composite materials. In particular, the zirconia particles of the present invention are useful for applications where volumetric shrinkage upon curing of the composite material would otherwise be undesirable or even prohibitive.

More particularly, the present invention provides a composite material comprising one or more fillers (including the zirconia particles defined herein) and a polymerizable resin base.

A particular feature of the present invention is that the martensitic transformation of the zirconia particles can be provoked by a trigger mechanism.

Thus, in a preferred embodiment of the composite material, the resin base, upon polymerization and in the absence of any compensating effect from the zirconia particles, causes a volumetric shrinkage (ΔVᵣₑₛᵢₙ) of the composite material of at least 0.50%, and wherein said composite material, upon polymerization of said resin base and upon phase transformation of said zirconia particles, exhibits a total volumetric shrinkage (ΔVₜₒₜₐₗ) of at least 0.25%-point less than the uncompensated volumetric shrinkage (ΔVᵣₑₛᵢₙ) caused by the resin base. More particularly, the volumetric shrinkage (ΔVᵣₑₛᵢₙ) is at least 1.00%, such as at least 1.50%, and the total volumetric shrinkage (ΔVₜₒₜₐₗ) is at least 0.50%-point less, such as 1.00%-point less than the uncompensated volumetric shrinkage.

The composite material typically comprises 5-95%, or 10-90%, by weight of the one or more fillers (including the zirconia particles) and 5-95%, or 10-90%, by weight of the polymerizable resin base, in particular 30-95%, or 30-90%, by weight of the one or more fillers and 5-70%, or 10-70%, by weight of the polymerizable resin base.

Calculated by volume, the composite material typically comprises 20-80% by volume of the one or more fillers (including zirconia particles) and 20-80% by volume of the polymerizable resin base, such as 25-80%, or 25-75%, by volume of the one or more fillers and 25-75% by volume of the polymerizable resin base.

Preferably, the composite material is substantially solvent free and water free. By the term “substantially solvent free and water free” is meant that the composite material comprises less than 1%, such as less than 0.5% or less than 150 ppm, by weight of solvents and/or water.

Alternatively, the present invention provides a composite material comprising one or more fillers (including zirconia particles) and a polymerizable resin base, wherein said one or more fillers comprises metastable zirconia in the tetragonal or cubic crystalline phase, wherein said resin base, upon polymerization and in the absence of any compensating effect from the zirconia particles, causes a volumetric shrinkage (ΔVᵣₑₛᵢₙ) of the composite material of at least 0.50%, and wherein said composite material, upon polymerization of said resin base and upon phase transformation of said filler ingredient(s), exhibits a total volumetric shrinkage (ΔVₜₒₜₐₗ) of at least 0.25%-point less than the uncompensated volumetric shrinkage (ΔVᵣₑₛᵢₙ) caused by the resin base, and wherein said material further comprises one or more water- or acid-releasing agents.

It is apparent that the one or more fillers, and in particular the zirconia particles, are important constituents of the composite material. Fillers are generally described above under “Fillers/Filler ingredients”.

The one or more fillers comprise at least one filler ingredient which (for the purpose of this section) at least include the zirconia particles. The term “filler ingredient” is intended to mean the filler or a fraction of the filler having particular physical properties, namely the inherent ability to compensate (by expansion) for volumetric shrinkage caused by polymerization and curing of the resin base.

The zirconia particles typically constitute(s) 20-100% of the total weight of the one or more fillers, e.g. 30-100%, such as 40-100% or 50-100%.

When calculated on the basis of the total weight of the composite material, the zirconia particles typically constitute(s) 15-90% of the total weight of the composite material, e.g. 25-90%, such as 30-90%, more specifically 60-85%.

Another important constituent of the composite material is the polymerizable resin base which is described in detail under “Polymerizable resin base”.

The composite material may comprise other constituents as disclosed under “Other constituents of the composite material”.

The population of zirconia particles is particularly useful in connection with dental filling material, see, e.g., under “Dental filling materials”. The general use of the population of zirconia particles in composite materials is described above under “Use of the composite materials”.

The initiation of martensitic transformation of the population of zirconia particles by means of application of ultrasound can advantageously be combined with the curing of the resin base by means of ultrasound, see, e.g., under “Combined initiation of martensitic transformation and curing of resin base by means of ultrasound”.

### Examples

The phase transformation is measured with the use of powder XRD. The volume fraction of monoclinic zirconia Vₘ can be determined from the following relationships:

Xₘ=(Iₘ(111)+Iₘ(11−1)/(Iₘ(111)+Iₘ(11−1)+Iₜ(111))

Vₘ=1.311 Xₘ/(1+0.311Xₘ)

Where Iₘ(111) and Iₘ(11−1) are the line intensities of the (111) and (11−1) peaks for monoclinic zirconia and Iₜ(111) is the intensity of the (111) peak for tetragonal zirconia.

### Example 1

Metastable tetragonal zirconia particles were subjected to normal air and phase transformed by the water content in less than 1 minute. With the use of XRD it was determined that the 70% of the tetragonal zirconia were phase transformed to the monoclinic phase.

### Example 2

Metastable tetragonal zirconia particles were subjected to a 1.5 M HCl solution in iso-propanol and phase transformed by the HCl content. With the use of XRD it was determined that the 60% of the tetragonal zirconia were phase transformed to the monoclinic phase.

### Example 3

Metastable tetragonal zirconia particles were subjected to a 1.5 M HCl solution in water and phase transformed by the water and HCl content. With the use of XRD it was determined that the 90% of the tetragonal zirconia were phase transformed to the monoclinic phase.

### Example 4

A test composite material is prepared by mixing 65 vol % of the zirconia particles to be tested and 35 vol % of a polymer resin system (36% (w/w) BisGMA, 43% (w/w) UDMA, 19.35% (w/w) TEGDMA. 0.3% (w/w) camphorquinone (CQ), 0.3% (w/w) N,N-dimethyl-p-amino-benzoic acid ethylester (DABE), 0.05% (w/w) 2,6-di-tert-butyl-4-methylphenol (BHT) and 1% (w/w) o-hydroxybenzyl ethanol. The phase transformation is initiated by UV-radiation, simultaneously with the curing of resin with light (blue).

### Example 5

A test composite material is prepared by mixing 65 vol % of the zirconia particles to be tested and 35 vol % of a polymer resin system (36% (w/w) BisGMA, 43% (w/w) UDMA, 19.350% (w/w) TEGDMA. 0.3% (w/w) camphorquinone (CQ), 0.3% (w/w) N,N-dimethyl-p-amino-benzoic acid ethylester (DABE), 0.05% (w/w) 2,6-di-tert-butyl-4-methylphenol (BHT) and 1% (w/w) 1-chloroanthraquinone). The phase transformation is initiated by UV-radiation, simultaneously with the curing of resin with light (blue).

### Example 6

A test composite material is prepared by mixing 65 vol % of the zirconia particles to be tested and 35 vol % of a polymer resin system (36% (w/w) BisGMA, 43% (w/w) UDMA, 19.35% (w/w) TEGDMA. 0.3% (w/w) camphorquinone (CQ), 0.3% (w/w) N,N-dimethyl-p-amlno-benzoic acid ethylester (DABE), 0.05% (w/w) 2,6-di-tert-butyl-4-methylphenol (BHT), 0.8% (w/w) diphenyliodonium hexafluorophosphate and 0.2% acridine dye). The phase transformation is initiated by light, simultaneously with the curing of resin.

### Example 7

A test composite material was prepared by mixing 200 mg of the zirconia particles to be tested and 500 mg of a polymer resin system (36% (w/w) BisGMA, 43% (w/w) UDMA, 19.35% (w/w) TEGDMA. 0.5% (w/w) camphorquinone (CQ), 0.5% (w/w) N,N-dimethyl-p-aminobenzoic acid ethylester (DABE), 0.05% (wlw)) with 50 mg of the trigger molecule (2-(4-Methoxystyryl)-4,6-bis(trichloromethyl)-1,3,5-triazine). The phase transformation was initiated by light from a curing device at max. intensity 1100 mW/cm² (Bluephase from Ivoclar Vivadent), simultaneously with the curing of resin. After 2 minutes 15% of the zirconia particles were phase transformed. After 30 min. 53% of the zirconia particles were phase transformed.

### Example 8

In a glove-box with a water content of <10 ppm water 210 mg of the zirconia particles and 150 mg Ph₂ICl were weighted into a glass flask. Then 21 g. MeOH was added with a magnetic stirring bar. The suspension was exposed to 22 hours of UV (9 W). The suspension was then filtered on a paper filter and dried in vacuum. The powder was cured in a dental resin (36% (w/w) BisGMA, 43% (w/w) UDMA, 19.35% (w/w) TEGDMA. 0.5% (w/w) camphorquinone (CQ), 0.5% (w/w) N,N-dimethyl-p-aminobenzoic acid ethylester (DABE), 0.05% (w/w)). The phase transformation was measured to 40%.

1. A composite material comprising one or more fillers and a polymerizable resin base, wherein said one or more fillers comprise at least one filler ingredient, said filler ingredient(s) being present in a metastable first phase and being able to undergo a martensitic transformation to a stable second phase, the volume ratio between said stable second phase and said metastable first phase of said filler ingredient(s) being at least 1.005, and wherein said material further comprises one or more water- or acid-releasing agents.

2. A composite material comprising one or more fillers and a polymerizable resin base, wherein said one or more fillers comprise at least one filler ingredient, said filler ingredient(s) including metastable zirconia in the tetragonal or cubic crystalline phase, wherein said resin base, upon polymerization and in the absence of any compensating effect from the one or more filler ingredients, causes a volumetric shrinkage (ΔVᵣₑₛᵢₙ) of the composite material of at least 0.50%, and wherein said composite material, upon polymerization of said resin base and upon phase transformation of said filler ingredient(s), exhibits a total volumetric shrinkage (ΔVₜₒₜₐₗ) of at least 0.25%-point less than the uncompensated volumetric shrinkage (ΔVᵣₑₛᵢₙ) caused by the resin base, and wherein said material further comprises one or more water- or acid-releasing agents.

3. The composite material according to claim 1, which is a dental filling material.

4. The dental filling material according to claim 3, wherein the filler ingredient(s) of the composite material include(s) zirconia (ZrO₂) in metastable tetragonal or cubic crystalline phase.

5. The dental filling material according to claim 4, consisting of:
40-85% by weight of the one or more fillers, wherein said one or more fillers comprise at least one filler ingredient, said filler ingredient(s) include(s) metastable zirconia in the tetragonal or cubic crystalline phase;
15-60% by weight of the a polymerizable resin base, said resin base being based on one or more compound selected from the group consisting of methacrylic acid (MA), methylmethacrylate (MMA), 2-hydroxyethyl-methacrylate (HEMA), triethyleneglycol dimethacrylate (TEGDMA), bisphenol-A-glycidyl dimethacrylate (BisGMA), bisphenol-A-propyl dimethacrylate (BisPMA), urethane-dimethacrylate (UDMA), and HEMA condensed with butanetetracarboxylic acid (TCB);
0.01-5% by weight of water-releasing agents;
0-5% by weight of additives; and
0-4% by weight of solvents and/or water.

6. The composite material according to claim 1, wherein the one or more water- or acid-releasing agents comprises at least one water-releasing agent.

7. The composite material according to claim 1, wherein the one or more water- or acid-releasing agents comprises at least one acid-releasing agent.

8. The composite material according to claim 1, wherein the one or more water- or acid-releasing agents is a combination at least one water-releasing agent and at least one acid-releasing agent.

9. A method of controlling the volumetric shrinkage of a composite material upon curing, comprising the step of:
(a) providing a composite material comprising one or more fillers and a polymerizable resin base, wherein said one or more fillers comprise at least one filler ingredient, said filler ingredient(s) being present in a metastable first phase and being able to undergo a martensitic transformation to a stable second phase, the volume ratio between said stable second phase and said metastable first phase of said filler ingredient(s) being at least 1.005, and wherein said material further comprises one or more water- or acid-releasing agents;
(b) allowing the resin base to polymerize and cure, and allowing the filler ingredient(s) to undergo a martensitic transformation from said first metastable phase to said second stable phase.

10. The method according to claim 9, wherein the composite material is as defined in claim 4.

11. The composite material according to claim 1, wherein the water- or acid-releasing agent(s) comprise(s) at least one triazine compound, said triazine compound comprising one or two trihalomethyl groups represented by the following general formula (I): 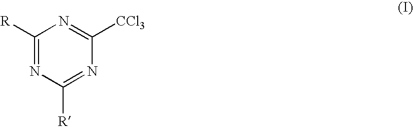
wherein CCl₃ may be replaced by a CF₃ group; R represents the attachment point for an organic moiety; and R is selected from the group consisting of a hydrogen atom, a further trihalomethyl group, a substituted C₁₋₆-alkyl group, an unsubstituted C₁₋₆-alkyl group, a substituted aryl group, an unsubstituted aryl group, and a substituted C₂₋₆-alkenyl group.

12. A composite material as defined in claim 1 for use in medicine.

13. The composite material according to claim 2, which is a dental filling material.

14. The dental filling material according to claim 13, wherein the filler ingredient(s) of the composite material include(s) zirconia (ZrO₂) in metastable tetragonal or cubic crystalline phase.

15. The dental filling material according to claim 14, consisting of:
40-85% by weight of the one or more fillers, wherein said one or more fillers comprise at least one filler ingredient, said filler ingredient(s) include(s) metastable zirconia in the tetragonal or cubic crystalline phase;
15-60% by weight of the a polymerizable resin base, said resin base being based on one or more compound selected from the group consisting of methacrylic acid (MA), methylmethacrylate (MMA), 2-hydroxyethyl-methacrylate (HEMA), triethyleneglycol dimethacrylate (TEGDMA), bisphenol-A-glycidyl dimethacrylate (BisGMA), bisphenol-A-propyl dimethacrylate (BisPMA), urethane-dimethacrylate (UDMA), and HEMA condensed with butanetetracarboxylic acid (TCB);
0.01-5% by weight of water-releasing agents;
0-5% by weight of additives; and
0-4% by weight of solvents and/or water.

16. The composite material according to claim 2, wherein the one or more water- or acid-releasing agents comprises at least one water-releasing agent.

17. The composite material according to claim 2, wherein the one or more water- or acid-releasing agents comprises at least one acid-releasing agent.

18. The composite material according to claim 2, wherein the one or more water- or acid-releasing agents is a combination at least one water-releasing agent and at least one acid- releasing agent.

19. The composite material according to claim 2, wherein the water- or acid-releasing agent(s) comprise(s) at least one triazine compound, said triazine compound comprising one or two trihalomethyl groups represented by the following general formula (I): 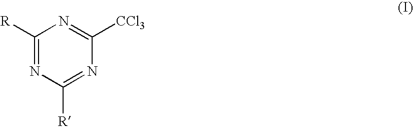
wherein CCl₃ may be replaced by a CF₃ group; R represents the attachment point for an organic moiety; and R is selected from the group consisting of a hydrogen atom, a further trihalomethyl group, a substituted C₁₋₆-alkyl group, an unsubstituted C₁₋₆-alkyl group, a substituted aryl group, an unsubstituted aryl group, and a substituted C₂₋₆-alkenyl group.
